(19) United States
(12) Patent Application Publication
(10) Pub. No.: US 2009/0258087 A1
(43) Pub. Date: Oct. 15, 2009

(54) Method to increase and accelerated rehabilitate the capacity for work

(75) Inventors: Igor Nikolaevich Roshchin, Moscow (RU); Sergey Alexandrovich Naumov, Tomsk (RU); Alexander Nikolaevich Orlov, Moscow (RU); Grigoriy Lukich Butakov, Moscow (RU); Vladimir Petrovich Smetannikov, Moscow (RU); Olga Alexandrovna Makarova, Moscow (RU)

Correspondence Address: THE NATH LAW GROUP, 112 South West Street, Alexandria, VA, 22314, US

(73) Assignee: ZAKRYTOE AKTSIONERNOE OBSHCHESTVO"ATOM-MED CENTER", Moscow (RU)

(21) Appl. No.: 11/991,168

(22) PCT Filed: Sep. 1, 2005
(86) PCT No.: PCT/RU2005/000445

§ 371 Date: Feb. 28, 2008

(51) Int. Cl. A61K 33/00

(52) U.S. Cl. 424/613

(57) ABSTRACT

The method to increase and accelerated rehabilitate the capacity for work is characterized in that the person is exposed to gaseous products having the property to improve physical and psychological human capabilities during a period of preparing for extreme strains and/or after extreme strains; the exposure to gaseous products is performed during a period from several weeks to several minutes before undergoing extreme strains by inhaling the gaseous mixture composed of oxygen and/or air and at least one inert gas, mainly xenon, with the contents of oxygen or air from 20% to 98% and at least one inert gas from 2% to 80%. The inhalation with the gaseous mixture is carried out until the state of euphoria emerges but no longer than for 20 minutes with breaks or without them; preferably, the inhalation with the gaseous mixture is performed no less than for 10 seconds and no longer than for 20 minutes with breaks or without them. Permanent and accelerated rehabilitation of the organism's capabilities and work capacity improvement during preparatory periods for extreme strains and in the course of extreme stressful strains as well as prompt rehabilitation of the diminished organism after extreme physical strains is provided due to the impact of gaseous products, i.e. oxygen cocktails having the property to improve human physical and psychological abilities and relieve mental, emotional and physical strain, pain and tiredness.

### FIELD OF THE INVENTION

This invention relates to medicine and can be used in sports medicine for setting records as well as in extreme medicine and rehabilitation practice for improving functional abilities and human body resistance at adaptive tension. Unregulated emotional stress in somatically and mentally healthy people leads, through activation of hypothalamo-pituitary and adrenal systems, to formation of a general adaptation syndrome related to energy consumption phases. This is confirmed by an increase in the situational anxiety level. Intensity of changes in the situational anxiety level, adaptive reactions and hormonal status grows in a pro-gradient way to the period of action of the stressing stimulant and remains at the maximum level for 24 hours of its aftereffect.

The invention can be used in the most efficient way under conditions of situational anxiety when sportsmen are getting ready for competitions, students are preparing for examinations, postgraduate students are preparing to defend their theses, and pilots are getting ready for combat flights, as well as for rehabilitation of the work capacity for people belonging to the above-mentioned categories as well as people engaged in physically demanding labor. Moreover, the method can be used in cases of performance and professional reliability impairment and changes in the psychophysiological state related to the overstrain of homeostatic structures and deterioration of compensation abilities in divers, stuntmen, miners, etc. under extreme conditions, in particular, at physical exercises. The invention can also be used for rehabilitation under clinical conditions, at physical exhaustion, electrolytic disorders, water depletion, to reduce side effects in case of treatment with antibiotics and chemotherapeutic drugs, under unfavorable ecological conditions (increased radiation, atmospheric pollution, etc.) and to accelerate acclimatization. In particular, the method is indicated for prompt rehabilitation after an intensive physical (muscular) or psychological overstrain. To embody the invention, it is expedient to make oxygen cocktails out of the mixture of inert gases and oxygen.

### PRIOR ART

A method to increase and rehabilitate the capacity for work in sportsmen including peroral introduction of the combined polyvitaminic drug named Supradin, one pill b.i.d. for 3-4 weeks during the training period and for 2-3 days during the period of competitions, has been known. (1)

Disadvantages of this method are as follows: it requires much time to obtain an apparent result; it is inefficient, does not enable to reduce the rehabilitation period after intensive physical activity and needs correction with other drugs on a permanent basis.

A method to increase and rehabilitate the capacity for work when people are subjected to gaseous products having the property to improve physical and psychological abilities due to negative hydroaeroionization of the gaseous medium during the period of preparation for extreme strains has been known. (2)

Disadvantages of this method are as follows: long-term process, low efficacy and complexity for being embodied under conditions when natural salt caves are unavailable. It does not enable to reduce the rehabilitation period after intensive physical activity and needs correction with other drugs on a permanent basis.

### SUMMARY OF THE INVENTION

The technical objective of the invention is to broaden the range of means to improve and accelerated rehabilitate the capacity for work by developing an efficient and safe method that can be used without having to suspend the training cycle.

The technical result providing a solution for the given objective lies in increasing the rate of impact to achieve the increased work capacity indices, simplifying the body of actions to embody the method, simplifying the equipment used, enabling to apply the gaseous product that can be stored for a long time, ensuring the comfortable state of the person exposed to the impact, providing a harmless combination with any types of food and drugs, expanding possible conditions of the use, excluding a negative impact on human body, accelerating rehabilitation after extreme strains and expanding possibilities for selecting the composition and dosage of the gaseous mixture on an individual basis as well as the inhalation mode taking into account the individual condition of the organism, expanding the range of conditions to be subjected to an efficient correction: disorders in the mental and emotional sphere (stresses, depressions, mental and emotional strains); general overstrain syndrome; abrupt change in climatic and geographic conditions including those after a change in the time-zone-related regimen (for example, when moving to competitions taking place in another hemisphere); muscular tone disorders (muscular hypertonia, myalgia); disorders of the functional state of sportsmen from senior age-related groups; consequences of accelerated weight reduction.

The essence of the invention in accordance with the first variant is in that a method to increase and accelerated rehabilitate the capacity for work when the person is exposed to gaseous products having the property to improve human physical and psychological abilities, characterized in that the exposure to gaseous products is conducted for a period from several weeks to several minutes before undergoing an extreme strain by means of inhalation with a gaseous mixture composed of oxygen and/or at least one inert gas, mainly xenon, with the contents of oxygen or air from 20% to 98% and at least one inert gas from 2% to 80%.

Preferably, the inhalation with the gaseous mixture is performed until the state of euphoria emerges but no longer than for 20 minutes with breaks or without them.

Preferably, the inhalation with the gaseous mixture is performed for no less than 10 seconds and no longer than for 20 minutes with breaks or without them.

Preferably, the inhalation with the gaseous mixture composed of oxygen or air from 20% to 49.99% and at least one inert gas from 50.01% to 80% is performed.

Preferably, the inhalation with the gaseous mixture composed of oxygen or air from 95.01% to 98% and at least one inert gas from 2% to 4.99% is performed.

Preferably, the inhalation with the gaseous mixture is performed again after completion of the extreme strain during a time interval from several minutes to several weeks.

Preferably, the inhalation with the gaseous mixture is performed again after completion of the extreme strain during a time interval that is less than 30 minutes.

Preferably, the inhalation with the gaseous mixture is performed again after completion of the extreme strain during a time interval that is more than 50 minutes but no more than several weeks.

Preferably, the inhalation with the gaseous mixture is performed using a mask of an anesthesia apparatus.

Preferably, the inhalation with the gaseous mixture is performed using an inhalator including portable or hand-held one.

Preferably, the inhalation is performed using the volume of the gaseous mixture that is more than 0.1 l but no more than 0.2 l per 1 kg of the body mass.

Preferably, the concentration of gases within the mixture is varied during the inhalation.

Preferably, each gas is fed from cylinders separately and gases are mixed directly during the inhalation.

Preferably, the premixed gaseous mixture is supplied from one cylinder during the inhalation.

Preferably, the inhalation with the gaseous mixture is performed when a person is subjected to long-term extreme physical and psychological strains.

Preferably, the inhalation with the gaseous mixture is performed in combination with phytotherapy.

Preferably, the inhalation with the gaseous mixture is performed in combination with special-purpose relaxing music.

As a rule, when embodying the method, sportsmen (patients) are exposed to gaseous mixtures for 2-7 days, in some cases for up to 14 days, depending on the functional and mental condition of their organism. There may be from one to seven inhalations for one patient. Gaseous mixtures are composed of inert gases (xenon is the basic one) and oxygen combined with other inert gases or without them. The composition depends on the objectives of rehabilitation (treatment). Inhalations are carried out with mixtures of various compositions and concentrations: xenon 2-80% and/or other inert gases 2-80%; oxygen 20-98%. Air can be used instead of oxygen. Mixtures are prepared directly during the inhalation by mixing gases from different cylinders or a premix of inert gases with oxygen and/or air (made within one cylinder) in different proportions can be used (to avoid hypoxia, there must be no less than 20% of oxygen in the mixture). Gases are mixed in proportions depending on the indications for achieving the curative effect.

The procedure is carried out using a mask of an anesthesia apparatus or inhalator including a portable or hand-held one from several seconds to 10-20 minutes with breaks or without them. To maintain the condition of the patient subjected to inhalation at the required level and to supply the therapeutic mixture to the patient, it is often necessary to adjust the concentration of inert gases and oxygen in the course of inhalation (depending on the indications). The volume of the gaseous mixture to be used for one procedure is not to exceed 0.02-0.2 l per 1 kg of the body mass.

The possibility to embody the technical result relates to normalization of the neuroendocrinal regulation of vital processes; muscle relaxing, pain reliving and anesthetic properties of inert gases, mainly, of xenon, as a part of the gaseous mixture with oxygen and/or air, which is an oxygen cocktail; prolonged action, their chemical inertness and their properties to be promptly redistributed in lungs and organism and also be excreted fast, to be combined with any drugs as well as stabilize hemodynamics, improve gas exchange, increase glucose level in peripheral blood, decrease insulin cortisol and hypophyseal hormones level by 20-30% and have a direct impact on serotonin release. The impact provided by the claimed method promotes an active (in 30-60 minutes) change in the hormonal status characterized by the decrease in the chondrotropic hormone and cortisol level. The decrease in the level of cortisol produced by adrenal glands that destroys proteins, fats and carbohydrates in case of a stress (extreme strain) enables to achieve accelerated rehabilitation of the balanced state of the organism.

Inhalation does not suppress the emotional and conative sphere of the patient. The procedure evokes positive emotions in patients and does not cause black vision.

### DETAILED DESCRIPTION OF THE INVENTION

### Example 1

A sportsman aged 26 who had the time limit of 4 min. 05 sec. when swimming at a distance of 400 m (his personal record). Body mass: 65.5 kg. Place of residence: Northeast of Russia. Place of competitions: Australia. The sportsman moved to Australia 2 days before the competitions. He had uneasy dreams before the race: he woke up three times, the reactive anxiety level by the Spielberg-Khanin scale made up 38, i.e. it was high. The sportsman was inhaling a mixture of xenon 35%, helium 10%, molecular oxygen 45% and air (the rest of the mixture contents) with the help of a mask of the Polynarcon-2P anesthesia apparatus for 2-5 minutes 6 hours before the test race until signs of light euphoria, relaxation, moderate pupil dilatation and horizontal nystagmus emerged. The procedure took 10.95 l of the mixture. Then there was a 400 m race with the time limit of 4 min. 01 sec. The race was repeated in a day with the result of 4 min. 02 sec. The sportsman had good dreams without any signs of anxiety. The reactive anxiety level by the Spielberg-Khanin scale made up 27, which is an average value.

In general, it was established that the anxiety level decreased, work capacity improved and dream normalized no later than in 10 minutes upon the inhalation with the aforesaid mixture, which was expressed in the race time limit improvement by 4 seconds. Signs of the extreme strain the sportsman had underwent were absent in the state of his health and hormonal status in a day after the inhalation, i.e. therapeutic effects of a single inhalation with a gaseous mixture of inert gases (both one-component and multi-component one) and oxygen/air maintain positive changes in compensatory and adaptive reactions characterized by improvement of physiologically feasible phases of the general adaptation syndrome and active formation of emotional stability 24 hours later.

### Example 2

Resistance to stress factors during long-term routes (naupathia as well as static and dynamic physical strain) and initial condition of the visual analyzer were determined in a group of truck drivers (8 people)—practically healthy men aged 27-48 with the body mass of 56-110 kg—whose condition was to a greater or smaller extent characterized by the general overstrain syndrome upon inhalation with a mixture of xenon 60%, helium 10%, neon 5% and oxygen 25% during 10-20 seconds 60 minutes before they go on route. The consumption of the inert gases mixture with molecular oxygen made up 2.8 l per person. The resistance to naupathia improved after the inhalation by 2.5 times on average; the “reserve time”on route increased by 1.5 times on average; the positive effect retained within 15±2 days.

Inhalations also improved spare capacities of the external respiration apparatus and physical efficiency: the lung capacity increased by 5-10%, maximum breathing capacity increased by 12-18%, the breath-holding time improved by 15-25% and the muscle activity performance increased by 14%. The functional level of the visual analyzer improved as well: the visual capacity improved by 10-12% while the threshold of brightness sensitivity decreased by 20-45%. All drivers under examination filled in self-assessment questionnaires during the experiment that were analyzed later. Seven experimental results out of eight turned out to be high.

### Example 3

There were two groups (control and test group) including 6 people each composed of metro tunnel workers (men aged 24-45 with the body mass 55-74.6 kg) with muscular hypertonia and myalgia who were exposed to inhalations with a mixture of xenon 2% and oxygen 40% (the rest of the mixture included air) within 19.5 minutes with the help of a mask-type apparatus. The consumption of the mixture of xenon with oxygen and air made up 8.35 l per person on average that prevented a transfer to the stage of pure anesthesia.

Inhalations were carried out at the preventorium twice a day (60 minutes before the shift and 15 minutes after it) every day during 14 days.

The control group was subjected to the procedure of introduction of phytoaerons being a fine electrically charged aerosol—an aqueous emulsion of essential oils—through inhalation.

The efficacy of the compared methods as for the work capacity improvement was assessed before and after the course of procedures by the generally accepted technique (4)—the physical efficiency test with variable load power in watts.

As a result, the following data were obtained: the efficiency improvement by 7.0%, which made up 16.4 Wt on average, was discovered in the control group (that was subjected to phytoaerons inhalations) after a course of procedures. The efficiency improvement by 19.8%, which made up 47.5 Wt on average, was discovered in the test group (that was subjected to inhalations with a mixture of xenon with oxygen/air) after a course of procedures. The prevalent reaction to extreme strains in the test group after several procedures was that of training proceeding with less apparent strain and demanding less metabolic costs from the organism as compared to regular activation, strain and stress reactions; not a single case of an emerging stress syndrome or individual anxiety formation was discovered.

### Example 4

Three sportsmen from senior age-related groups (long-distance runners) suffered from disorders of their functional state accompanied by intense pain and spasms of leg muscles during intensive training. They received inhalations with a mixture of xenon 4% and oxygen 96% within 10 min. ±15 sec. each every day for two weeks before the test competitions beginning with the third day of the training period (without suspending their physical activity). The consumption of the mixture of xenon with oxygen made up 9.1 l per person on average. Muscle pain disappeared in two sportsmen and greatly reduced in one sportsman. Muscles got relaxed; sportsmen started feeling warmth and tingling in them. Initially, the anesthetic effect of inhalation lasted in two sportsmen until the following morning; in one sportsman it lasted for five hours upon inhalation. Muscle spasms did not resume. Inhalations had a longer effect (more than a day) in three days.

Sportsmen showed results that matched their personal records during competitions.

After competitions, the aforesaid inhalations lasted for 6 min. ±15 sec. for 7 days with the consumption of the xenon and oxygen mixture making up 7.1 l per person on average. Physiological parameters and readiness for further competitions restored completely for the aforesaid period. The rehabilitation period used to take no less than 15 days.

### Example 5

A fighter (29 years old) with the body mass of 67.2 kg. His physical activity deteriorated, he had uneasy dreams and bad appetite and felt irritation after losing weight before important competitions. He received inhalations with a mixture of xenon 40%, krypton 40% and oxygen 20% on a daily basis for 6 min. ±15 sec. for one week before the competitions. The consumption of the xenon and oxygen mixture made up 10.1 l on average and resulted in the state of euphoria.

Irritation was arrested, and dream and appetite were normalized in a day after inhalations. The sportsman had good physical fitness in 7 days, by the time of the competitions.

The fighter demonstrated results complying with his maximum capabilities during the competitions.

The aforesaid inhalations resumed and lasted for 25 days after the competitions within 3 min. ±15 sec. with the consumption of the xenon and oxygen mixture making up 5.5 l per person on average. Physiological parameters and readiness for further competitions restored completely for the said period. The rehabilitation period used to take no less than 30 days.

### Example 6

Mental and emotional strain—a stress-related state characterized by absence of motivation to train and asthenization of the body—developed in a sportsman aged 31 (track and field athletics) during a period between competitions. Triple increase of cortisol and prolactin and moderate increase of creatinine phosphokinase as well as insubstantial reduction of the total protein was discovered in her blood tests. Myocardium perfusion was found based on her ECG results. The body mass made up 56.5 kg.

She received inhalations with oxygen cocktail mixture composed of xenon 80% and oxygen 20% for 5 min. ±15 sec. on a daily basis for 5 days before the competitions. Inhalations were accompanied with special-purpose relaxing music.

The consumption of xenon and oxygen made up 7 l on average, thus providing the state of euphoria.

The stress state was arrested, and dream and appetite normalized in the first day after the inhalations. Her condition improved the next day; she was in a good mood and was eager to train again. Her dream and appetite became normal. Her physical form restored on the following days. Control blood tests showed a tendency to normalization of stress hormones, and ECG became normal in three days. She had a good physical condition before the competitions. She won a prize at a Russian championship.

Xenon 2% and oxygen 98% inhalations were carried out for 14 days after the competitions. They lasted for 5 min. ±15 sec. with the consumption of the xenon and oxygen mixture amouting to 6.2 l on average. Physiological parameters and readiness for further competitions restored completely for the said period. The rehabilitation period used to take no less than 25 days.

### Example 7

A sportsman aged 21, a sprinter. Body mass: 65.5 kg. He had a series of trainings with long-term intensive strains lasting for 14 days. His condition was characterized by general physical tiredness, loss of dream and appetite and general irritation. There were complaints about muscular pain and stiffness. The sportsman was inhaling a mixture of xenon 50%, helium 10%, argon 5%, krypton 5% and molecular oxygen 10% and air (the rest of the mixture contents) with the help of a mask of the Polynarcon-2P anesthesia apparatus for 3-10 minutes for the following 4 days until signs of euphoria, relaxation, moderate pupil dilatation and horizontal nystagmus emerged. Inhalations were conducted in combination with drug plants phytotherapy. It took 7.75 l of the mixture. Then there was a 100 m race for 11 seconds. The race took place again in a day with the result of 12 seconds. Dream and appetite were good; anxiety was absent; the reactive anxiety level by the Spielberg-Khanin scale made up 28, i.e. it was medium.

It was established that the dream normalized, anxiety level decreased, efficiency improved no later than in 20 minutes upon the inhalation with the aforesaid mixture, which was expressed in getting a race time that was close to a record one. Signs of the extreme strain the sportsman had underwent were absent in the hormonal status and state of his health in 24 hours after the inhalation.

Thus, as a result of this invention the range of means to accelerated rehabilitate the work capacity and improve the work capacity was broadened and an efficient and safe method that can be used without having to suspend the training cycle was developed.

As a result of this, permanent and accelerated rehabilitation of the organism's capabilities and work capacity improvement during periods of preparation for extreme strains and in the course of extreme stressful strains as well as prompt rehabilitation of the diminished organism after extreme physical strains is provided due to the impact of gaseous products, i.e. oxygen cocktails having the property to improve human physical and psychological abilities and relieve mental, emotional and physical strain, pain and tiredness.

### INDUSTRIAL APPLICATIONS

This invention is embodied with the use of multifunctional and easily available up-to-date equipment and substances that are widely used in the industry and health care.

Information sources:

1. R. D. Seifullina, I. A. Ankudinova. The dope monster. Farmakologiya Sporta, Moscow, 1996, p. 153.

2. RU No. 20043112, 1995.

3. SU No. 1776401, 1992 (prototype).

4. Instrumental methods of studying the cardiovascular system, under the editorship of T. Ye. Vinogradova, Moscow, Meditsina, 1986, pp. 397-401.

1-17. (canceled)

18. A method to increase and accelerated rehabilitate the capacity for work when the person is exposed to gaseous products having the property to improve human physical and psychological abilities, characterized in that the exposure to gaseous products is conducted for a period from several weeks to several minutes before undergoing an extreme strain by means of inhalation with a gaseous mixture composed of oxygen and/or at least one inert gas, mainly xenon, with the contents of oxygen or air from 20% to 98% and at least one inert gas from 2% to 80%, and inhalation with the gaseous mixture is performed until the state of euphoria emerges for no less than 10 seconds, inhalation is performed using the volume of the gaseous mixture that is more than 0.1 litre but no more than 0.2 liter per 1 kg of the body mass.

19. The method according to claim 18, characterized in that the inhalation with the gaseous mixture is performed no longer than for 20 minutes with breadks or without them.

20. The method according to claim 18, characterized in that the inhalation with the gaseous mixture composed of oxygen or air from 20% to 49.99% and at least one inert gas from 50.01% to 80% is performed.

21. The method according to claim 18, characterized in that the inhalation with the gaseous mixture composed of oxygen or air from 95.01% to 98% and at least one inert gas from 2% to 4.99% is performed.

22. The method according to claim 18, characterized in that the inhalation with the gaseous mixture is performed again after completion of the extreme strain during a time interval from several minutes to several weeks.

23. The method according to claim 22, characterized in that the inhalation with the gaseous mixture is performed again after completion of the extreme strain during a time interval that is less than 30 minutes.

24. The method according to claim 22, characterized in that the inhalation with the gaseous mixture is performed again after completion of the extreme strain during a time interval that is more than 50 minutes but no more than several weeks.

25. The method according to claim 18, characterized in that the inhalation with the gaseous mixture is performed using a mask of an anesthesis apparatus.

26. The method according to claim 18, characterized in that the inhalation with the gaseous mixture is performed using an inhalator including portable or hand-held one.

27. The method according to claim 18, characterized in that the concentration of gases within the mixture is varied during the inhalation.

28. The method according to claim 18, characterized in that each gas is fed from cylinders separately and gases are mixed directly during the inhalation.

29. The method according to claim 18, characterized in that a premixed gaseous mixture is supplied from one cylinder during the inhalation.

30. The method according to claim 18, characterized in that the inhalation with the gaseous mixture is performed when a person in subjected to long-term extreme physical and psychological strains.

31. The method according to claim 18, characterized in that the inhalation with the gaseous mixture is performed in combination with phytotherapy.

32. The method according to claim 18, characterized in that the inhalation with the gaseous mixture is performed in combination with special-purpose relaxing music.
